(19) United States
(12) Patent Application Publication
(10) Pub. No.: US 2018/0000827 A1
(43) Pub. Date: Jan. 4, 2018

(54) PHARMACEUTICAL COMPOSITION COMPRISING GEFITINIB

(71) Applicant: SYNTHON B.V., Nijmegen (NL)

(72) Inventors: Deepak MURPANI, Nijmegen (NL); Marta VIVANCOS MARTINEZ, Sant Boi de Llobregat (ES); Lisardo ALVAREZ FERNANDEZ, Sant Boi de Llobregat (ES); Luis NOGUEIRAS NIETO, Sant Boi de Llobregat (ES)

(21) Appl. No.: 15/537,778

(22) PCT Filed: Dec. 16, 2015
(86) PCT No.: PCT/EP2015/080019

§ 371 Date: Jun. 19, 2017

(30) Foreign Application Priority Data
Dec. 19, 2014 (EP) PCT/EP2014/078800

(51) Int. Cl. A61K 31/5377; A61K 9/28

(52) U.S. Cl. CPC A61K 31/5377; A61K 9/284

(57) ABSTRACT

The present invention relates to pharmaceutical tablet compositions comprising the compound gefitinib as the active pharmaceutical ingredient and a PVA-based coating, suitable for oral administration as well as to processes for their preparation. The compositions exhibit good long-term stability and dissolution properties.

The invention relates to pharmaceutical tablet compositions comprising the compound gefitinib as the active pharmaceutical ingredient, suitable for oral administration.

### BACKGROUND OF THE INVENTION

Gefitinib, chemically 4-(3-chloro-4-fluorophenylamino)-7-methoxy-6-[3-(4-morpholinyl)propoxy] quinazoline of formula (1)

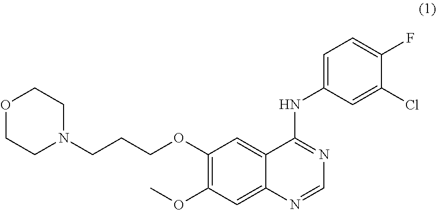 [SMILES: COc1cc2ncnc(Nc3ccc(F)c(Cl)c3)c2cc1OCCCN1CCOCC1]

is a pharmaceutically active compound acting as a selective inhibitor of epidermal growth factor receptor's (EGFR) tyrosine kinase. Gefitinib is used as a medicament for the treatment of e.g., locally advanced or metastatic non-small-cell lung cancer (NSCLC), and is available, e.g., under the brand name Iressa®, as 250 mg tablets for oral administration.

Gefitinib was generically disclosed in EP556226. Specifically, gefitinib and its salts were disclosed in EP 823900, whereas specific polymorphic forms of gefitinib base were disclosed in EP1480650 (form 1, anhydrate and form 5, trihydrate) and WO2006/090413 (form 6, monohydrate).

Furthermore, EP823900 generically discloses many possible pharmaceutical compositions, e.g., oral tablets or capsules, prepared in a conventional manner using conventional excipients. Example 32 (a) to (c) provide several possible tablet compositions, which may optionally be enteric coated with e.g., a cellulose acetate phthalate.

Various pharmaceutical tablet compositions comprising gefitinib, which may serve for purposes of oral administration of gefitinib to a patient in need thereof, have been disclosed in prior art documents.

Gefitinib is a weak alkaline compound and has two basic groups with pKa values of approximately 5.3 and 7.2. Consequently, the solubility of gefitinib is highly dependent upon pH. Changing pH values in the gastro-intestinal tract has a high effect on the absorption of gefitinib.

EP1480679 discloses a tablet composition comprising gefitinib and a water soluble cellulose ether or an ester thereof. The presence of the claimed excipients significantly reduces the rate of precipitation of gefitinib from solutions with pH values similar to either that of the stomach or the upper gastro-intestinal tract. In the marketed 250 mg tablets, water soluble cellulose ether hydroxypropyl methylcellulose (HPMC) is present only in small amounts in the coating layer. The core of the composition of claim 15 comprises from 45 to 55% of gefitinib, from 25 to 40% lactose, from 5 to 15% microcrystalline cellulose, from 2 to 6% disintegrant, from 1 to 5% povidone, from 0.05 to 1% sodium lauryl sulphate and from 0.1 to 4% lubricant (magnesium stearate). The film coating comprises from 0.5 to 3% water-soluble cellulose ether, from 0 to 0.5% plasticizer, from 0 to 0.5% dispersion aid, from 0 to 0.5% opacifier and 0 to 0.5% colorant.

To reduce the pH sensitivity of gefitinib and thus to provide a more uniform delivery at high concentrations of gefitinib to the major absorption sites in the gastro-intestinal tract, the compositions of EP1651233 comprise both a water-soluble cellulose ether or an ester of a water-soluble cellulose ether and a water-soluble acid. Examples are given with e.g. HPMC and an acid, e.g. fumaric or malic acid, in the tablet core.

CN102631347A discloses a tablet composition comprising 20-65% gefitinib, 20-75% diluent, 0.1-3% solubiliser, 1-5% binding agent, 2-10% disintegrant and 0.4-2% lubricant. The tablets are prepared by dissolving the binding agent, solubiliser and gefitinib in an acidic solution and adding this solution to the diluent and disintegrant, followed by pelletizing and tabletting.

The dispersible gefitinib tablet of CN102266300A comprises 10-65% gefitinib, 10-30% diluent, 10-50% disintegrant, 5-60% acidifier, 0.1-20% binder and 0.1% lubricant and flow aid, wherein the filler may be e.g. starch, lactose, and/or microcrystalline cellulose; the disintegrant may be e.g., croscarmellose sodium, and/or crospovidone; the acidifier may be e.g., citric acid, malic acid, and/or fumaric acid; the binder may be e.g., hydroxypropyl methylcellulose, and/or povidone; and the lubricant and flow aid may be silica powder, and/or magnesium stearate.

In WO2010/081443 complexes of gefitinib with water soluble tectons are prepared in order to avoid polymorphic conversion of the gefitinib base. These complexes are used for the preparation of pharmaceutical compositions. The tectons may be low-molecular, i.e., mono- and oligosaccharides with 1 to 9 monomeric units, ascorbic acid, vitamins A and E, amino acids, guanidine and its derivatives, urea, thiourea, aminosaccharides, amides of aliphatic and aromatic acids, sulfonamides, surfactants-excipients, such as tween 80. The tectons may also be polymeric, i.e., alginic acids, pectins, alginic acid, polysaccharide carboxylic and sulfonated systems, beta glucan, ester pectins, polygalacturonic acid, sulfonated dextrans, chitosan salts, PEGylated chitosan, PVP, PEG, Pluronic, polylactides, and polylactides-polyglycolides.

In summary, the prior art teachings indicate that in order to enhance the bioavailability of gefitinib either a water-soluble cellulose ether or an ester of a water-soluble cellulose ether is needed in the formulation. Preferably a water-soluble acid is also present. Furthermore, to prevent the gefitinib base from polymorphic conversion, it may be necessary to prepare a complex with a tecton before preparing the tablet composition. Thus, it will be beneficial to provide an alternative tablet composition for oral administration of gefitinib, which shows good stability without polymorphic conversion of the gefitinib base and provides good bioavailability.

### SUMMARY OF THE INVENTION

The present invention relates to a pharmaceutical tablet composition suitable for oral administration of gefitinib, which composition exhibits good stability upon long-term storage without polymorphic conversion of the gefitinib base and provides good bioavailability.

In one aspect, the present invention relates to a pharmaceutical tablet composition comprising from 45-60% of gefitinib, from 10-50% of a water soluble or water insoluble diluent or combinations thereof, from 0.5-5% of a binding agent, from 0.1-5% of a wetting agent, from 2-6% of a disintegrant, and from 0.4-4% of a lubricant, wherein the pharmaceutical tablet composition comprises a PVA-based coating.

In one embodiment, the pharmaceutical tablet composition comprises from 45-55% of gefitinib, from 25-40% of lactose, from 5-15% of microcrystalline cellulose, from 1-3% of povidone, from 0.1-1% of sodium lauryl sulphate, from 3-5% of croscarmellose sodium, and from 0.5-2% of magnesium stearate.

The pharmaceutical tablet composition may be prepared by an aqueous wet granulation process, an organic granulation process or a dry granulation process. Most preferably, an aqueous wet granulation process is used.

The PVA-based coating of the pharmaceutical tablet composition is selected from Opadry® II and Kollicoat®.

The amount of PVA-based coating is 2-7 wt % of the tablet core weight.

Preferably, the gefitinib used in the pharmaceutical tablet composition has a particle size distribution characterised by a D₉₀ of less than about 40 μm.

The tablet composition is preferably packed in Triplex/Alu or Alu/Alu blister pack material.

Preferably, the pharmaceutical tablet composition comprises from 50 to 500 mg of gefitinib, more preferably 250 mg of gefitinib, calculated as the free base.

In a second aspect, the invention relates to the use of the pharmaceutical tablet composition of the invention as a medicament for treating NSCLC.

### DETAILED DESCRIPTION OF THE INVENTION

The present invention relates to a pharmaceutical tablet composition, suitable for oral use, comprising gefitinib. “Gefitinib” is a generically used name for4-(3-chloro-4-fluorophenylamino)-7-methoxy-6-[3-(4-morpholinyl)propoxy] quinazoline and will be so used throughout this specification, unless expressly stated differently.

Solid state gefitinib may exist as a crystalline or an amorphous material. Crystalline materials may exist in different polymorphic modifications. In addition, they may be substantially anhydrous (e.g., form 1) or may exist in the form of a hydrate (e.g., monohydrate form 6 or trihydrate form 5) and/or a solvate (e.g., methanol solvate form 2 or DMSO solvate form 3). Any such modifications are included within the terms “gefitinib” throughout this specification.

Gefitinib is either commercially available or may be obtained by processes known in the art.

The essential features of the pharmaceutical tablet compositions of the present invention are from 45-60% of gefitinib, from 10-50% of a water soluble or water insoluble diluent or combinations thereof, from 0.5-5% of a binding agent, from 0.1-5% of a wetting agent, from 2-6% of a disintegrant, and from 0.4-4% of a lubricant, wherein the pharmaceutical tablet composition comprises a polyvinyl alcohol (PVA)-based coating.

EP1480679 teaches that in order to enhance the bioavailability of gefitinib either a water-soluble cellulose ether or an ester of a water-soluble cellulose ether is needed in the formulation. According to EP1651233 and CN102631347A, preferably a water-soluble acid is also present, either in the composition or in the production process. Furthermore, to prevent the gefitinib base from polymorphic conversion, it may be necessary to prepare a complex with a tecton before preparing the tablet composition as described in WO2010/081443.

Surprisingly, it was found that simple tablet compositions of gefitinib may be prepared without the addition of water soluble cellulose ethers or esters thereof through either a process of aqueous wet granulation, organic granulation or dry granulation. In addition, the tablets of the present invention do not have to be prepared in an acidic environment. Surprisingly, it was found that the tablet compositions of the present invention, comprising a PVA-based coating, show no increase in the rate of precipitation of gefitinib from solutions with pHs similar to either that of the stomach or the upper gastro-intestinal tract when compared with the marketed 250 mg Iressa® tablets with HPMC coating. Furthermore, the compositions of the present invention exhibit excellent long-term stability without polymorphic conversion of the gefitinib base.

Accordingly, the present invention provides for a pharmaceutical tablet composition for oral administration comprising gefitinib.

Water-soluble diluents advantageously comprise polyols and sugars such as mannitol, sorbitol, lactose, etc., but are not limited thereto. The preferred water-soluble diluents is lactose.

Water-insoluble diluents advantageously comprise a cellulose, a cellulose derivative and a starch, but are not limited thereto. Preferably the water insoluble diluent is microcrystalline cellulose or starch or a combination thereof.

Binding agents advantageously comprise povidone and polyethylene glycol, but are not limited thereto. Preferred binder is povidone.

Wetting agents advantageously comprise sodium lauryl sulphate and polysorbate 80, but are not limited thereto. Preferred wetting agent is sodium lauryl sulphate.

Disintegrants advantageously comprise croscarmellose sodium, crospovidone, starches, sodium starch glycolate, and clays, but are not limited thereto. Preferred disintegrant is croscarmellose sodium.

Lubricants advantageously comprise magnesium stearate and sodium stearyl fumarate, but are not limited thereto. Preferred lubricant is magnesium stearate.

In one advantageous embodiment, the pharmaceutical tablet composition comprising a PVA-based coating comprises from 45-55% of gefitinib, from 25-40% of lactose, from 5-15% of microcrystalline cellulose, from 1-3% of povidone, from 0.1-1% of sodium lauryl sulphate, from 3-5% of croscarmellose sodium, and from 0.5-2% of magnesium stearate.

The pharmaceutical tablet composition of the invention is preferably a swallowable tablet. The dosage form advantageously comprises a unit dose of gefitinib, which may be from 50 to 500 mg of gefitinib, preferably 250 mg of gefitinib, calculated as the free base.

The tablet compositions of the present invention display dissolution behavior typical for immediate-release formulations, exhibiting a dissolution rate of at least 85% in 10 minutes when tested in 1000 ml 0.01 N aqueous HCl pH 1.0, 0.01 N aqueous HCl pH 2.0, acetate buffer pH 4.5 or 5% Tween in water in a USP apparatus II at 50 rpm. The dissolution profiles of the tablets are similar to the profiles of Iressa®.

The pharmaceutical tablet composition of the present invention may be prepared by

(a) An aqueous wet granulation process;

(b) An organic granulation process; or

(c) A dry granulation process.

Preferably, the gefitinib has a particle size distribution characterised by a D₉₀ of less than about 40 μm. The known particle size analysis methods can be used for determining the particle size, for example particle size measurement using the Malvern Mastersizer aqueous dispersion method with a low amount of a suitable surfactant and moderate sonication.

Gefitinib may optionally be milled and/or pre-screened before mixing in order to remove lumps. Advantageously, the particles of the treated product pass a screen with 400-800 μm (0.4-0.8 mm) mesh size. Accordingly, the various excipients may be treated in the same manner.

Sub a)

Gefitinib, the water soluble and/or water insoluble diluent(s) and the disintegrant (complete or partial quantity) are blended and sieved through a sieve of a suitable mesh size. The binder and wetting agent are dissolved in a sufficient amount of purified water to make a binder solution. The binder solution is added to the blend and a granulate is formed in a high shear granulator, followed by drying and milling to a suitable size. The obtained granules are mixed with the remaining quantity of disintegrant and lubricated. The mix is compressed using suitable punches.

Sub b)

Organic granulation avoids water and is therefore effective in solving polymorphic issues arising due to water (e.g., formation of dihydrates, trihydrates, etc.).

Gefitinib, the water soluble and/or water insoluble diluent(s) and the disintegrant (complete or partial quantity) are blended and sieved through a sieve of a suitable mesh size. The binder and wetting agent are dissolved in a sufficient amount of ethanol or isopropyl alcohol (IPA) to make a binder solution. The binder solution is added to the blend and a granulate is formed in a high shear granulator, followed by drying and milling to a suitable size. The obtained granules are mixed with the remaining quantity of disintegrant and lubricated. The mix is compressed using suitable punches.

Sub c)

Dry granulation is effective in solving polymorphic conversion as the granulation occurs without any aqueous or organic solvent.

Gefitinib, the water soluble and/or water insoluble diluent(s), the binder, the wetting agent and the disintegrant (complete or partial quantity) are blended and sieved through a sieve of a suitable mesh size. The blend is mixed with a partial quantity of the lubricant. The blend is then compacted in a roller compactor or slugged using suitable slug punches, followed by milling to a suitable size. The obtained granules are mixed with the remaining quantity of disintegrant and lubricated with the remaining quantity of lubricant. The mix is compressed using suitable punches.

The preferred process to prepare the tablets of the present invention is by an aqueous wet granulation process.

The tablet compositions are coated with a PVA-based coating. Coating is done using any conventional coating technique known in the art, such as spray coating in a conventional coating pan or fluidized bed processor; or dip coating. Preferably, the pharmaceutical composition of the current invention is coated with a PVA-based coating selected from Opadry® II, wherein polyvinyl alcohol (PVA) is the base polymer and Kollicoat®, which is a polyvinyl alcohol-polyethylene glycol graft copolymer.

Preferably the tablet compositions of the present invention comprise 2-7 wt % of the tablet core weight.

Based on laboratory experiments, prior art and non-conventional packaging of the marketed 250 mg Iressa® tablets (PVC-blisters packed within aluminium pouch pack), it is clear that gefitinib and its tablet formulation are sensitive to chemical, physical and/or polymorphic stability. Gefitinib base is reported to exist in various hydrated forms, i.e., anhydrous form 1, monohydrate form 6 and trihydrate form 5.

The pharmaceutical compositions of the present invention are packaged in blister pack material. The blister pack materials to be used in accordance with the present invention may be any blister pack material known to a person of ordinary skill in the art. Suitable blister pack materials to be used in accordance with the present invention are selected from the group of PVC/Alu, Duplex/Alu, Triplex/Alu and Alu/Alu. To ensure protection of the compositions of the present invention from e.g. moisture and thereby preventing polymorphic conversions, Triplex/Alu and Alu/Alu are particularly preferred blister pack materials.

The pharmaceutical tablet compositions of the present invention are useful, for treating a disease or condition treatable by gefitinib. Thus, in a second particular aspect, the present invention relates to a pharmaceutical tablet composition comprising gefitinib according to the present invention for use as a medicament, preferably for treating various cancer indications, more preferable for treating NSCLC.

The invention will be further illustrated by the following non-limiting examples.

### EXAMPLES

### Examples with Aqueous Wet Granulation

### Example 1

### Aqueous Wet Granulation with Water Soluble and Insoluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Lactose (anhydrous or | 72.5-90 | Water soluble | |
| monohydrate | | diluent | |
| Microcrystalline | 72.5-90 | Water insoluble | |
| cellulose (MCC) | | diluent | |
| Povidone (K-30) | 4.5-9.0 | Binder | |
| Sodium lauryl sulphate | 5.0-15 | Wetting agent/ | |
| (SLS) or polysorbate 80 | | surfactant | |
| Purified water | q.s. | For binder | |
| | | solution | |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose, MCC and a partial quantity of disintegrant (e.g. 50%) are blended and sieved through a sieve of a suitable mesh size. Povidone and SLS or povidone and polysorbate 80 are dissolved in a sufficient amount of purified water to make a binder solution. The binder solution is added to the blend and a granulate is formed in a high shear granulator, followed by drying and milling to a suitable size. The obtained granules are mixed with the remaining quantity of disintegrant and lubricated with magnesium stearate. The mix is compressed using suitable punches.

### Example 2

### Aqueous Wet Granulation with Water Insoluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Microcrystalline cellulose | 145-180 | Water insoluble | |
| (MCC) or starch, or a | | diluent | |
| combination thereof | | | |
| Povidone (K-30) | 4.5-9.0 | Binder | |
| Sodium lauryl sulphate | 5.0-15 | Wetting agent/ | |
| (SLS) or polysorbate 80 | | surfactant | |
| Purified water | q.s. | For binder | |
| | | solution | |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose, MCC and/or starch and a partial quantity of disintegrant (e.g. 50%) are blended and sieved through a sieve of a suitable mesh size. Povidone and SLS or povidone and polysorbate 80 are dissolved in a sufficient amount of purified water to make a binder solution. The binder solution is added to the blend and a granulate is formed in a high shear granulator, followed by drying and milling to a suitable size. The obtained granules are mixed with the remaining quantity of disintegrant and lubricated with magnesium stearate. The mix is compressed using suitable punches.

### Example 3

### Aqueous Wet Granulation with Water Soluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Lactose (anhydrous or | 145-180 | Water soluble | |
| monohydrate or mannitol | | diluent | |
| Povidone (K-30) | 4.5-9.0 | Binder | |
| Sodium lauryl sulphate | 5.0-15 | Wetting agent/ | |
| (SLS) or polysorbate 80 | | surfactant | |
| Purified water | q.s. | For binder | |
| | | solution | |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose or mannitol and a partial quantity of disintegrant (e.g. 50%) are blended and sieved through a sieve of a suitable mesh size. Povidone and SLS or povidone and polysorbate 80 are dissolved in a sufficient amount of purified water to make a binder solution. The binder solution is added to the blend and a granulate is formed in a high shear granulator, followed by drying and milling to a suitable size. The obtained granules are mixed with the remaining quantity of disintegrant and lubricated with magnesium stearate. The mix is compressed using suitable punches.

### Examples with Organic Granulation

Organic granulation avoids water and is therefore effective in solving polymorphic issues arising due to water (e.g., formation of dihydrates, trihydrates, etc.).

### Example 4

### Organic Granulation with Water Soluble and Insoluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Lactose (anhydrous or | 72.5-90 | Water soluble | |
| monohydrate | | diluent | |
| Microcrystalline | 72.5-90 | Water insoluble | |
| cellulose (MCC) | | diluent | |
| Povidone (K-30) | 4.5-9.0 | Binder | |
| Sodium lauryl sulphate | 5.0-15 | Wetting agent/ | |
| (SLS) or polysorbate 80 | | surfactant | |
| Ethanol or isopropyl | q.s. | For binder | To avoid |
| alcohol ((IPA) | | solution | polymorphic |
| | | | conversion |
| | | | of the active |
| | | | ingredient |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose, MCC and a partial quantity of disintegrant (e.g. 50%) are blended and sieved through a sieve of a suitable mesh size. Povidone and SLS or povidone and polysorbate 80 are dissolved in a sufficient amount of ethanol or IPA to make a binder solution. The binder solution is added to the blend and a granulate is formed in a high shear granulator, followed by drying and milling to a suitable size. The obtained granules are mixed with the remaining quantity of disintegrant and lubricated with magnesium stearate. The mix is compressed using suitable punches.

### Example 5

### Organic Granulation with Water Insoluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Microcrystalline | 145-180 | Water insoluble | |
| cellulose (MCC) or | | diluent | |
| starch | | | |
| Povidone (K-30) | 4.5-9.0 | Binder | |
| Sodium lauryl sulphate | 5.0-15 | Wetting agent/ | |
| (SLS) or polysorbate 80 | | surfactant | |
| Ethanol or isopropyl | q.s. | For binder | To avoid |
| alcohol ((IPA) | | solution | polymorphic |
| | | | conversion |
| | | | of the active |
| | | | ingredient |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose, MCC or starch and a partial quantity of disintegrant (e.g. 50%) are blended and sieved through a sieve of a suitable mesh size. Povidone and SLS or povidone and polysorbate 80 are dissolved in a sufficient amount of ethanol or IPA to make a binder solution. The binder solution is added to the blend and a granulate is formed in a high shear granulator, followed by drying and milling to a suitable size. The obtained granules are mixed with the remaining quantity of disintegrant and lubricated with magnesium stearate. The mix is compressed using suitable punches.

### Example 6

### Organic Granulation with Water Soluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Lactose (anhydrous or | 145-180 | Water soluble | |
| monohydrate or mannitol | | diluent | |
| Povidone (K-30) | 4.5-9.0 | Binder | |
| Sodium lauryl sulphate | 5.0-15 | Wetting agent/ | |
| (SLS) or polysorbate 80 | | surfactant | |
| Ethanol or isopropyl | q.s. | For binder | To avoid |
| alcohol ((IPA) | | solution | polymorphic |
| | | | conversion |
| | | | of the active |
| | | | ingredient |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose or mannitol and a partial quantity of disintegrant (e.g. 50%) are blended and sieved through a sieve of a suitable mesh size. Povidone and SLS or povidone and polysorbate 80 are dissolved in a sufficient amount of ethanol or IPA to make a binder solution. The binder solution is added to the blend and a granulate is formed in a high shear granulator, followed by drying and milling to a suitable size. The obtained granules are mixed with the remaining quantity of disintegrant and lubricated with magnesium stearate. The mix is compressed using suitable punches.

### Examples with Dry Granulation (Roller Compaction/Slugging)

Dry granulation is effective in solving polymorphic conversion as the granulation occurs without any aqueous or organic solvent.

### Example 7

### Dry Granulation with Water Soluble and Insoluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Lactose (anhydrous or | 72.5-90 | Water soluble | |
| monohydrate | | diluent | |
| Microcrystalline | 72.5-90 | Water insoluble | |
| cellulose (MCC) | | diluent | |
| Povidone (K-30) | 4.5-9.0 | Binder | |
| Sodium lauryl sulphate | 5.0-15 | Wetting agent/ | |
| (SLS) | | surfactant | |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose, MCC, povidone, SLS and a partial quantity of disintegrant (e.g. 50%) are blended and sieved through a sieve of a suitable mesh size. Mix the blend with a partial quantity of magnesium stearate. The blend is then compacted in a roller compactor or slugged using suitable slug punches, followed by milling to a suitable size. The obtained granules are mixed with the remaining quantity of disintegrant and lubricated with the remaining quantity of magnesium stearate. The mix is compressed using suitable punches.

### Example 8

### Dry Granulation with Water Insoluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Microcrystalline | 145-180 | Water insoluble | |
| cellulose (MCC) or | | diluent | |
| starch | | | |
| Povidone (K-30) | 4.5-9.0 | Binder | |
| Sodium lauryl sulphate | 5.0-15 | Wetting agent/ | |
| (SLS) | | surfactant | |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, MCC, povidone, SLS and a partial quantity of disintegrant (e.g. 50%) are blended and sieved through a sieve of a suitable mesh size. Mix the blend with a partial quantity of magnesium stearate. The blend is then compacted in a roller compactor or slugged using suitable slug punches, followed by milling to a suitable size. The obtained granules are mixed with the remaining quantity of disintegrant and lubricated with the remaining quantity of magnesium stearate. The mix is compressed using suitable punches.

### Example 9

### Dry Granulation with Water Soluble Fillers

| | Per tablet | | |
|---|---|---|---|
| Ingredient | (mg) | Function | Remark |
| Gefitinib (form 1 or | 250 | Active | D₉₀ below |
| monohydrate form 6) | | ingredient | 40 μm |
| | | | D₅₀ below |
| | | | 20 μm |
| Lactose (anhydrous or | 145-180 | Water soluble | |
| monohydrate or mannitol | | diluent | |
| Povidone (K-30) | 4.5-9.0 | Binder | |
| Sodium lauryl sulphate | 5.0-15 | Wetting agent/ | |
| (SLS) | | surfactant | |
| Croscarmellose sodium/ | 11.0-22.0 | Disintegrant | |
| crospovidone/sodium | | | |
| starch glycolate | | | |
| Magnesium stearate | 4.5-9.0 | Lubricant | |
| Core tablet weight | 420-480 | | |

Process:

Gefitinib, lactose, povidone, SLS and a partial quantity of disintegrant (e.g. 50%) are blended and sieved through a sieve of a suitable mesh size. Mix the blend with a partial quantity of magnesium stearate. The blend is then compacted in a roller compactor or slugged using suitable slug punches, followed by milling to a suitable size. The obtained granules are mixed with the remaining quantity of disintegrant and lubricated with the remaining quantity of magnesium stearate. The mix is compressed using suitable punches.

### Example 10 to 18

Tablet cores of examples 1 to 9 were coated with 2-7% of a PVA-based coating.

### Example 19

### Aqueous Wet Granulation with Water Soluble and Insoluble Fillers and Opadry II Coating

| | | | |
|---|---|---|---|
| | | Per tablet | |
| Ingredient | % | (mg) | Function |

Tablet core

| | | | |
|---|---|---|---|
| Gefitinib (form 1), | 50.0 | 250.0 | Active ingredient |
| D₉₀ below 40 μm | | | |
| Lactose monohydrate | 32.7 | 163.5 | Water soluble |
| | | | diluent |
| Microcrystalline | 10.0 | 50.0 | Water insoluble |
| cellulose (MCC) | | | diluent |
| Croscarmellose sodium | 4.0 | 20.0 | Disintegrant |
| Povidone | 2.0 | 10.0 | Binder |
| Sodium lauryl sulphate | 0.3 | 1.5 | Wetting agent/ |
| (SLS) | | | surfactant |
| Magnesium stearate | 1.0 | 5.0 | Lubricant |
| Core tablet weight | | 500 | |

Coating

| | | | |
|---|---|---|---|
| Opadry ® II | 2.5 | 12.5 | Coating |

Process:

Gefitinib, lactose, MCC and croscarmellose sodium are blended and sieved through a sieve of a suitable mesh size. Povidone and SLS are dissolved in a sufficient amount of purified water to make a binder solution. The binder solution is added to the blend and a granulate is formed in a high shear granulator, followed by drying and milling to a suitable size. The obtained granules are mixed magnesium stearate. The mix is compressed using suitable punches. The tablets are coated with an Opadry® II coating.

The tablets obtained, exhibited a dissolution rate of at least 85% in 10 minutes when tested in 1000 ml 0.01 N HCl pH 1.0, 0.01 N HCl pH 2.0, acetate buffer pH 4.5 or 5% Tween in water in a USP apparatus II at 50 rpm. The dissolution profiles of the tablets were similar to the profiles of Iressa®.

### Example 20

### Aqueous Wet Granulation with Water Soluble and Insoluble Fillers and Kollicoat® Coating

| | | | |
|---|---|---|---|
| | | Per tablet | |
| Ingredient | % | (mg) | Function |

Tablet core

| | | | |
|---|---|---|---|
| Gefitinib (form 1), | 50.0 | 250.0 | Active ingredient |
| D₉₀ below 40 μm | | | |
| Lactose monohydrate | 32.7 | 163.5 | Water soluble |
| | | | diluent |
| Microcrystalline | 10.0 | 50.0 | Water insoluble |
| cellulose (MCC) | | | diluent |
| Croscarmellose sodium | 4.0 | 20.0 | Disintegrant |
| Povidone | 2.0 | 10.0 | Binder |
| Sodium lauryl sulphate | 0.3 | 1.5 | Wetting agent/ |
| (SLS) | | | surfactant |
| Magnesium stearate | 1.0 | 5.0 | Lubricant |
| Core tablet weight | | 500 | |

Coating

| | | | |
|---|---|---|---|
| Kollicoat ® | 2.5 | 12.5 | Coating |

Process:

Gefitinib, lactose, MCC and croscarmellose sodium are blended and sieved through a sieve of a suitable mesh size. Povidone and SLS are dissolved in a sufficient amount of purified water to make a binder solution. The binder solution is added to the blend and a granulate is formed in a high shear granulator, followed by drying and milling to a suitable size. The obtained granules are mixed magnesium stearate. The mix is compressed using suitable punches. The tablets are coating with an Opadry® II coating.

The tablets obtained, exhibited a dissolution rate of at least 85% in 10 minutes when tested in 1000 ml 0.01 N HCl pH 1.0, 0.01 N HCl pH 2.0, acetate buffer pH 4.5 or 5% Tween in water in a USP apparatus II at 50 rpm. The dissolution profiles of the tablets were similar to the profiles of Iressa®.

1. A pharmaceutical tablet composition comprising
from 45-60% of gefitinib;
from 10-50% of a water soluble or water insoluble diluent or combinations thereof;
from 0.5-5% of a binding agent;
from 0.1-5% of a wetting agent;
from 2-6% of a disintegrant; and
from 0.4-4% of a lubricant;
wherein the pharmaceutical tablet composition comprises a PVA-based coating.

2. The pharmaceutical tablet composition of claim 1 comprising
from 45-55% of gefitinib;
from 25-40% of lactose;
from 5-15% of microcrystalline cellulose;
from 1-3% of povidone;
from 0.1-1% of sodium lauryl sulphate;
from 3-5% of croscarmellose sodium; and
from 0.5-2% of magnesium stearate.

3. The pharmaceutical tablet composition according to claim 1 prepared by an aqueous wet granulation process, an organic granulation process or a dry granulation process.

4. The pharmaceutical tablet composition according to claim 1 prepared by an aqueous wet granulation process.

5. The pharmaceutical tablet composition according to claim 1 wherein the PVA-based coating is selected from Opadry® II and Kollicoat®.

6. The pharmaceutical tablet composition according to claim 1, wherein the amount of coating is 2-7 wt % of the tablet core weight.

7. The pharmaceutical tablet composition according to claim 1, wherein the gefitinib has a particle size distribution characterised by a D₉₀ of less than 40 μm.

8. The pharmaceutical tablet composition according to claim 1, packed in Triplex/Alu or Alu/Alu blister pack material.

9. The pharmaceutical tablet composition according to claim 1, comprising from 50 to 500 mg of gefitinib, calculated as the free base.

10. (canceled)

11. The pharmaceutical tablet composition according to claim 9, comprising from 250 mg of gefitinib, calculated as the free base.

12. The pharmaceutical tablet composition according to claim 1, wherein said PVA-based coating comprises a polymer selected from polyvinyl alcohol polymers and polyvinyl alcohol-polyethyleneglycol graft copolymers.
